(19) United States
(12) Patent Application Publication
(10) Pub. No.: US 2025/0114252 A1
(43) Pub. Date: Apr. 10, 2025

(54) Method of Manufacturing a Device for Detecting Moisture at an Absorbent Article

(71) Applicant: Pampett AB, Lund (SE)

(72) Inventor: Johan Sjöholm, Lund (SE)

(21) Appl. No.: 18/987,336

(22) Filed: Dec. 19, 2024

Related U.S. Application Data
(63) Continuation of application No. 17/049,255, filed on Oct. 20, 2020, now abandoned.

(30) Foreign Application Priority Data
Apr. 20, 2018 (SE) 1850463-9
Apr. 20, 2018 (SE) 1850464-7
Apr. 20, 2018 (SE) 1850465-4

(51) Int. Cl. A61F 13/42; A61F 13/15

(52) U.S. Cl. CPC A61F 13/42; A61F 2013/15056; A61F 2013/424

(57) ABSTRACT

A method of manufacturing a device for detecting moisture at an absorbent article arrangeable to be attached on the absorbent article is disclosed. The method comprising providing a moisture barrier to protect a resonance circuit while leaving a moisture sensitive part exposed.

### BACKGROUND

### Technical Field

The present concept generally relates to a method of manufacturing of a device for detecting moisture at an absorbent article. More specifically, the present concept relates to a method of providing part of a circuit with a barrier to protect it from moisture.

### BACKGROUND

Moisture detection is important in a number of different situations. One such situation is in relation to absorbent articles, such as diapers. In the prior art, there exists solutions for alerting e.g. nursing staff upon detection of moisture in a diaper of a wearer, whereby the nursing staff knows that it is time to change the diaper.

WO 2007/069945 discloses a wetness detecting means comprising an electrical circuit which is integrally formed into an absorbent article, such as a diaper. However, this wetness detecting means may be unpractical and uneconomical since it requires integration with the diaper and hence specially made diapers.

Thus, there is a need in the prior art for a convenient, reliable and economical solution for detecting the presence of a patient.

### SUMMARY

In view of the above, an objective of the present inventive concept is to provide an easy and economical way of manufacturing the devices, and particular when sealing part of the device from moisture.

According to a first aspect of the disclosure, a method of manufacturing a device for detecting moisture at an absorbent article arrangeable to be attached on the absorbent article is disclosed. The method provides a moisture barrier to protect the resonance circuit while leaving the moisture sensitive part exposed. The method comprising providing a plurality of circuits arranged side by side to form a string or strip of circuits. The plurality of circuits may be provided on a roll. Each circuit of the plurality of circuits may be a herein described device, and includes a resonance circuit and a moisture sensitive part formed by two conducting layers arranged on opposite sides of an insulating layer. The method further includes providing at least one strip of a moisture barrier material. The moisture barrier may be provided on a roll. Attaching the moisture barrier material to both sides of the plurality of circuits, so that the resonance circuit is covered by the moisture barrier material but not the moisture sensitive part of each circuits, and forming single devices by separating the covered circuits.

According to a further aspect of the disclosure, a system for detecting a presence of a patient is described. The system comprises a device for detecting moisture at an absorbent article arranged to be attached on the absorbent article. The device may comprise a resonance circuit including a moisture sensitive part, wherein the resonance circuit may have at least a first resonance frequency when the moisture sensitive part is in a dry condition and at least a second resonance frequency when the moisture sensitive part is in a moist condition. The system may further comprise a monitoring unit arranged for transmitting a test signal to the device, and determine if the patient is present based a response signal from the device.

In one example of the system may a lack of response signals detected by the monitoring unit be an indication that the patient is not present. Alternatively, that the detected signal is not one of the expected response signals from the device for detecting moisture at an absorbent article.

In one example of the system may the monitoring unit be configured for transmitting an alarm signal to a receiving unit based on a determination that the patient may not present. In some alternative and/or additional examples, the monitoring unit may transmit a signal to a receiving unit to indicate that the patient is present.

In one example of the system may the alarm signal start a surveillance system which may be configured for sending a verifying signal, such as an image signal and/or a sound signal, to the receiving unit.

In a further aspect of the disclosure is a method of detecting a presence of a patient described. The method comprising, arranging a moisture detecting device on an absorbent article. The device may comprise a resonance circuit including a moisture sensitive part. The resonance circuit may have a first resonance frequency when the moisture sensitive part is in a dry condition and a second resonance frequency when the moisture sensitive part is in a moist condition. The method further comprising, transmitting a test signal to the device, receiving a response signal from the device, and determining if the patient is present based on the response signal.

According to an aspect of the disclosure, there is provided a system for detecting moisture at an absorbent article, the system may comprise a device arranged to be attached on the absorbent article. The device may comprise a resonance circuit including a moisture sensitive part, wherein the resonance circuit has a first resonance frequency when the moisture sensitive part is in a dry condition and a second resonance frequency when the moisture sensitive part is in a moist condition. The system may further comprise a monitoring unit being arranged for transmitting a test signal to the device, receive a response signal from the device, determine a frequency of the response signal and generate a detection signal if the determined frequency corresponds to the second resonance frequency.

According to one example, the system may be arranged for detecting moisture at, on or in the absorbent article. The absorbent article may be any one of a diaper, an incontinence garment, a sanitary pad, a tampon a bandage, a bed protector or the like.

In this context, a dry condition may relate to a condition wherein no soiling at the device has occurred. Moreover, a moist condition may relate to a condition wherein soiling has occurred. The soiling may be release of moisture or fluid due to urination, evacuation of the bowel or other bodily waste.

By providing a device having a first and a second resonance frequency depending on the presence of moisture, moisture may be reliably detected. Moreover, by determining the frequency of the response signal the detection may be relatively insensitive to variations in the signal strength of the incoming signal e.g. due to varying distance between the device and the antenna or objects in the signal path dampening the test signal and the response signal.

Moreover, by generating the detection signal based on the presence of a signal at the second resonance frequency, generation of false detection signals due to absence of a device in an absorbent article may be avoided.

By utilizing that the detected response signal, such as lack of a response signal, may correlate with a patient not being present at a location within the range of the monitoring unit, a cheap and simple system may be provided for detecting the presents of a patient. Detecting the presence of a patient is important, for example during bed time when the patient is resting or sleeping and staff, such as care takers, are not present. Monitoring and determining the present of a patient may be especially be needed for patients diagnosed with dementia which could lead to disorientation, and confusion.

The described system provides a cheap and effective method for monitoring or determining the present of a patient without further equipment. By attaching the device on the absorbent article, the device may be used in a plurality of different absorbent articles with a minimum of adaption. For example, the device may be attached by sewing, stitching, adhesion or similar. Hence, the described system does not require specially made or customized absorbent articles. A further advantage is that the device may be provided on different locations of the absorbent article. The sensitivity of the detection may hence be varied on an individual basis. For example, in case the absorbent article is a diaper the device may be attached on different locations in the diaper for different wearers.

According to one example, the device comprises a fabric layer. The fabric layer may be provided as an outer layer on the device, facing the wearer of the absorbent article. This may increase the comfort for the wearer.

According to one example, the moisture sensitive part comprises an absorption layer arranged to absorb moisture. By absorbing the moisture, the moisture may be retained at the moisture sensitive part. The frequency shift from the first to the second resonance frequency may hence be maintained whereby moisture may be detected also a while after the moisture has been released in the absorbent article.

According to one example, the absorption layer includes a polymer.

Especially, the polymer may be a polyacrylic acid. According to one example, the device comprises a moisture barrier covering at least a portion of the resonance circuit. Especially, the moisture barrier does not cover at least a part of the moisture sensitive part. The moisture barrier prevents circuit parts other than the moisture sensitive part from coming into contact with moisture and thus prevents unpredictable resonance frequency shifts.

According to one example, the moisture sensitive part is arranged to shift the resonance frequency of the resonance circuit from the first resonance frequency to the second resonance frequency in the moist condition. Especially, the moisture sensitive part may provide this frequency shift by presenting a first capacitance in the dry condition and a second capacitance in the moist condition.

According to one example, the moisture sensitive part comprises two conductors separated by an insulator. The two conductors may be arranged in parallel. In the dry condition, the two conductors may provide a capacitive contribution to the resonance circuit. In the moist condition, fluid (e.g. water) and any charge carriers (e.g. ions) therein may lower the impedance between the two conductors. The resonance frequency may thereby be shifted from a first to a second resonance frequency.

According to one example, the two conductors form an interdigital finger structure. This structure provides a plurality of parallel conductors and thus enables a device with a large moist sensitive area and a high sensitivity to moist.

According to one example the device comprises an electrically insulating layer having a first side and a second side, wherein the two conductors are provided on the first side and the moisture sensitive part further comprises a third conductor which is provided on the second side opposite to the two conductors and is arranged to be capacity-coupled to the two conductors. The moisture sensitive part may thus present a first capacitance between the two conductors provided on the first side, a second capacitance between the third conductor and the first of the two conductors provided on the first side, and a third capacitance between the third conductor and the second of the two conductors provided on the first side. In a dry condition, the resonance frequency of the resonance circuit may thus be defined by the first capacitance connected in parallel with the series connected second and third capacitances and any further capacitances or inductances of the resonance circuit. However, in a moist condition the resonance frequency of the resonance circuit may be defined mainly by these further capacitances or inductances of the resonance circuit. As a result, a reliable shift from a first resonance frequency in a dry condition to a second resonance frequency in a moist condition may be achieved. This disclosed design of the device is particularly suitable for the frequency based moisture detection in accordance with the described concept.

According to one example, the device comprises a substance which is provided at the two conductors provided on the first side, the substance forming charge carriers when dissolved. These charge carriers may increase the sensitivity of the device. Especially, the substance may be a salt such as sodium chloride.

According to one example, the resonance circuit comprises an inductor and a capacitor,

According to one example, the test signal comprises at least one pulse. By transmitting a pulsed test signal, the power consumption of the monitoring unit may be reduced.

According to one example, the first resonance frequency is outside a frequency band or bandwidth of the test signal. According to this example, the device may respond strongly to the test signal in a moist condition. This may reduce the risk of false detections.

According to an alternative example, the first frequency is inside a frequency band or bandwidth of the test signal. According to this example, the device may respond to the test signal both in a moist and a dry condition. A shorter pulse length of the alternative example implies reduced power consumption of the monitoring unit.

According to one example, the system further comprises an antenna connected to the monitoring unit. The test signal is transmitted through the antenna. Additionally, the response signal may be received through the antenna. The antenna may thus be used both for transmitting the test signal and for receiving the response from the device. This may simplify handling of the system and reduce the costs of the system.

According to one example, the monitoring unit is arranged to reduce residual oscillations in the antenna, the oscillations generated by the test signal. This may facilitate detection of response signals from the device and increase the reliability of the moisture detection. According to one example, the monitoring unit is further arranged to determine an envelope of the response signal, and generate the detection signal if the envelope matches a reference envelope and if a determined frequency of the response signal corresponds to the second resonance frequency. If a received signal presents an expected envelope and has a frequency corresponding to the second resonance frequency, it may be determined that the received signal likely originates from the device and not from any other source. False detections due to noise at the second resonance frequency may hence be avoided.

According to a second aspect of the described concept, there is provided a method for detecting moisture at a device attached on an absorbent article, the device comprising a resonance circuit including a moisture sensitive part, the resonance circuit having a first resonance frequency when the moisture sensitive part is in a dry condition and a second resonance frequency when the moisture sensitive part is in a moist condition. The method comprising transmitting a test signal to the device, receiving a response signal from the device, determining a frequency of the response signal, and generating a detection signal if the determined frequency corresponds to the second resonance frequency.

The details and advantages discussed in relation to the first aspect apply correspondingly to the second aspect whereby reference hereby is made to the previous discussion.

### BRIEF DESCRIPTION OF THE DRAWINGS

The above, as well as additional objects, features and advantages of the present inventive concept, will be better understood through the following illustrative and non-limiting detailed description of preferred examples of the present described concept, with reference to the appended drawings, where like reference numerals will be used for like elements, wherein:

FIG. 1 illustrates a system for detecting moisture in a diaper where the system may be utilized for detecting the presence of a patient in accordance with an example of the described concept.

FIGS. 2a and b illustrate the upper side and the underside, respectively, of a device in accordance with an example of the described concept.

FIGS. 2c and d illustrate a resonance circuit in a dry condition and in a moist condition, respectively, in accordance with an example of the described concept.

FIGS. 3a and b illustrate the upper side and the underside, respectively, of a device in accordance with an alternative example of the described concept.

FIGS. 3c and d illustrate a resonance circuit in a dry condition and in a moist condition, respectively, in accordance with an alternative example of the described concept.

FIG. 4 is a flow chart of a method for detecting moisture which may be utilized for detecting the presence of a patient in accordance with an example of the described concept.

FIG. 5 is illustrating an implementation of the herein described circuit.

FIG. 6 is a flow chart of method of manufacturing a device for detecting moisture at an absorbent article arrangeable to be attached on the absorbent article.

FIG. 7 is illustrating a cross-section of a device manufactured according to the method herein described.

FIGS. 8a and 8b are illustrating an example of a mattress cover which includes an antenna. Channels for the antenna is also illustrated.

FIG. 9 is illustrating a further example of a mattress cover which includes an antenna.

### DETAILED DESCRIPTION OF EXAMPLES

The following examples will be described in relation to a diaper. However, the described concept is equally applicable also to other types of absorbent articles, e.g. incontinence garments, sanitary pads, tampons, bandages, or bed protectors or the like.

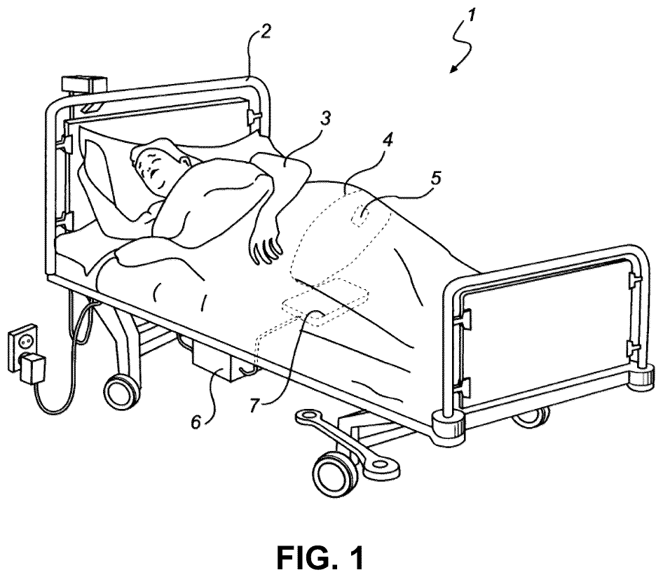

FIG. 1 illustrates a system 1 for detecting moisture in a diaper of a first example in accordance with the inventive concept. The system 1 of the first example will be illustrated in the context of a bed 2 carrying a patient 3 wearing a diaper 4. The system 1 comprises a device 5 for detecting moisture in the diaper 4. The system 1 further comprises a monitoring unit 6 and an antenna 7. The monitoring unit 6 is connected to the antenna 7. The detected signal from the device 5 by the monitoring unit 6, such as the lack of a signal, may be used to detect the presence of a patient at a location within the detection range of the monitoring unit 6. Alternatively, that the detected signal is not one of the expected response signals from the device 5.

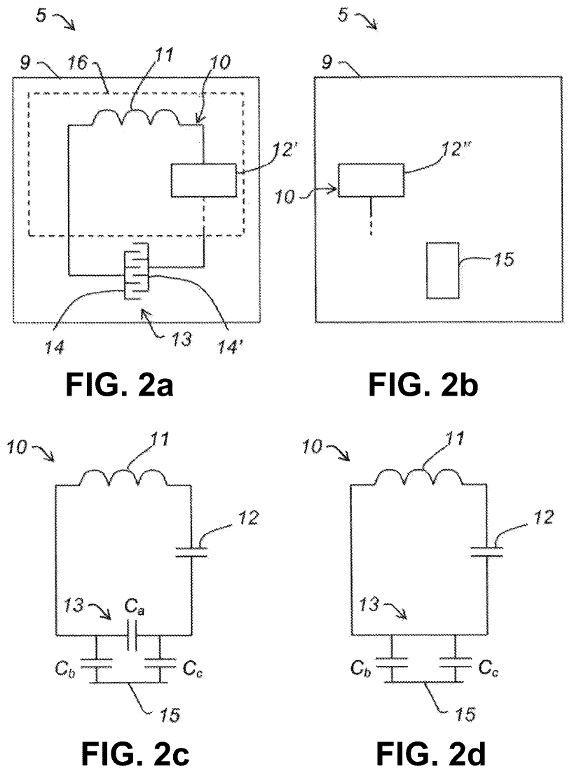

FIGS. 2a and b illustrate the device 5 in greater detail. The device 5 is provided in the form of a transponder tag. The device 5 comprises a flexible carrier having an adhesive underside (left out from the figures for increasing the clarity). The device 5 may hence be conveniently attached in the diaper 4. The device 5 is preferably attached at a location in the diaper such that the device 5 will become moist in case the patient 3 urinates or evacuates the bowel. The device 5 comprises an electrically insulating layer 9 such as a plastic film. The insulating layer 9 is provided on the upper side of the carrier. The insulating layer 9 may be coextensive with the carrier. The device 5 further comprises a resonance circuit 10 and a moisture sensitive part 13. The resonance circuit 10 and the moisture sensitive part 13 are provided on the insulating layer 9. The resonance circuit 10 may e.g. be formed by selectively etching off portions of a thin metallic layer provided on the insulating layer 9 according to principles well-known in the art.

With reference to FIGS. 2a-d, the resonance circuit 10 and the moisture sensitive part 13 will now be described in detail. The resonance circuit 10 comprises an inductor 11 and a capacitor 12. The inductor 11 is provided on a first side of the insulating layer 9. The capacitor 12 is formed by two conducting surfaces 12′, 12″ provided on opposite sides of the insulating layer 9. The inductor 11 is connected to the capacitor plate 12′ provided on the first side of the insulating layer 9. Preferably, the first side faces the wearer.

The device 5 further comprises a moisture barrier 16 covering the inductor 11 and the capacitor 12 of the resonance circuit 10. The moisture barrier 16 prevents moisture from coming into contact with the inductor 11 and the capacitor 12. In some examples, the moisture barrier 16 covers both opposite sides fully sealing the device 5 from moisture apart from the moisture sensitive part 13.

The device 5 further comprises an outer fabric layer (left out for increasing the clarity of the figure) covering the side of the device 5 facing the patient 3 to minimize skin irritation.

As illustrated in FIG. 2a, the moisture sensitive part 13 includes two sets of conductors 14 and 14′ provided on the first side of the insulating layer 9. The two sets of conductors 14, 14′ form an interdigital finger structure. The conducting fingers of the two sets of conductors 14, 14′ are provided in parallel to each other. The two sets of conductors 14 and 14′ are separated by an insulator, such as air or a dielectric. The number of fingers, the length of the fingers and the separation of the fingers may be varied depending on e.g. the specific application, the sensitivity of the device 5 or the size of the device 5 etc.

The moisture sensitive part 13 further comprises a conducting layer 15 provided on the second side of the insulating layer 9. The conducting layer 15 is provided opposite to the two sets of conductors 14, 14′. The conducting layer 15 thus directly faces at least a part of the two sets of conductors 14, 14′. The two sets of conductors 14 and 14′ are connected to the resonance circuit 10. More specifically, the first set of conductors 14 is connected to the inductor 11. The second set of conductors 14′ is connected to the capacitor plate 12″. The conducting layer 15 is not galvanically connected to the resonance circuit 10. By providing the conducting layer 15 opposite to the two sets of conductors 14 and 14′, the conducting layer 15 may be capacity-coupled to each of the sets of conductors 14 and 14′.

In case the two sets of conductors 14 and 14′ are dry, i.e. the moisture sensitive part is in a dry condition, there will be essentially no free charge carriers which may carry a current between the two sets of conductors 14 and 14′. As a result, the moisture sensitive part 13 forms a capacitor of capacitance Ca connected in parallel with two series connected capacitors of capacitance Cb and Cc, wherein Ca is the capacitance between the two sets of conductors 14 and 14′, Cb is the capacitance between the first set of conductors 14 and the conducting layer 15, and Cc is the capacitance between the second set of conductors 14′ and the conducting layer 15. This is illustrated in FIG. 2c.

Cg is proportional, among others, to the height extension of the two sets of conductors 14 and 14′ above the insulating layer 9 and inversely proportional to the separation between the fingers of the two sets of conductors 14 and 14′. Cb and Cc are proportional, among others, to the surface area of the conducting layer 15 and the first and second sets of conductors 14 and 14′, respectively, in the plane of the insulating layer 9 and inversely proportional to the separation between the conducting layer 15 and the first and second sets of conductors 14 and 14′ (i.e. the thickness of the insulating layer 9). According to the first example, the height extension is much smaller than the surface area and the thickness of the insulating layer 9 is much smaller than the separation between the two sets of conductors 14 and 14′. Consequently, Ca will be relatively small compared to C and Cc. As viewed from the resonance circuit 10, the moisture sensitive part 13 hence acts approximately as two series connected capacitances Cb and Cc. Thus, in a dry condition the resonance frequency of the resonance circuit 10 is mainly defined by the inductance of the inductor 1 1 connected in series with three series connected capacitances, namely the capacitance of the capacitor 12 and the capacitances Cb and Cc.

When the device 5 comes into contact with moisture, the moisture will penetrate the outer fabric layer and come into contact with the two sets of conductors 14 and 14′. Any charge carriers present in the fluid may then give rise to current conducting paths between the two sets of conductors 14 and 14′. The impedance between the two sets of conductors 14 and 14′ will hence decrease. If a sufficient amount of charge carriers is present, the moisture sensitive part 13 will present a low impedance current path between the two sets of conductors 14 and 14′ in parallel with Cb and Cc. This is illustrated in FIG. 2d in which the capacitance Ca has been replaced by a short circuit.

Thus, in a sufficiently moist condition the resonance circuit 10 will present a resonance frequency which is mainly defined by the inductance of the inductor 1 1 connected in series with the capacitance of the capacitor 12. This resonance frequency will be lower than the resonance frequency of the resonance circuit 10 in the dry condition. Hence, as the moisture sensitive part 13 is exposed to moisture the resonance frequency of the resonance circuit 10 will be shifted from a first resonance frequency to a second resonance frequency which is lower than the first resonance frequency.

It is to be understood that “short circuit” is a relative expression since in practice any conducting path will present some degree of resistance for a current. Thus, in this context the expression “short circuit” relates to a condition wherein the capacitances Cb and Cc in the moisture sensitive part 13 no longer influences the resonance frequency of the resonance circuit 10 appreciably.

The sensitivity of the device 5, may be increased by providing a salt (or other substance with similar properties in presence of moist) at the two sets of conductors 14 and 14′. When the salt comes into contact with fluid, it will dissolve and form charge carriers which may contribute to decrease the impedance between the two sets of conductors 14 and 14′.

Optionally, an absorption layer may be provided at the two sets of conductors 14 and 14′. The absorption layer may include a polymer such as a polyacrylic acid. The absorption layer may retain fluid released into the diaper 4 and thereby increase the sensitivity of the device 5.

According to a further option, the absorption layer may include a salt (or other substance with similar properties in presence of moist) to further increase the sensitivity of the device 5. By way of example, a composition of 40% polymer and 60% salt to 60% polymer and 40% salt may be appropriate proportions in some cases.

Returning to the first example, the resonance circuit 10 assumes a first resonance frequency when the moisture sensitive part 3 is in a dry condition and a second resonance frequency, which is different from the first resonance frequency, when the moisture sensitive part 13 is in a moist condition.

By transmitting a resonant electromagnetic signal to the resonance circuit 10, the resonance circuit 10 will start to resonate or oscillate at its resonance frequency. The resonance circuit 10 will continue to oscillate for a while also after electromagnetic signal has been stopped. This oscillation will generate an electromagnetic sinusoidal response signal with an exponentially decreasing amplitude envelope. This response signal may be picked-up by the antenna 7 and detected by the monitoring unit 6.

A moist condition may hence be determined based on the frequency of the response signal. Preferably, the “detection frequency” is chosen as the second resonance frequency. However, depending on the application and the desired sensitivity of the moisture detection, the detection frequency may also be chosen as a frequency between the first and the second resonance frequency.

To facilitate moist detection, the first resonance frequency and the second resonance frequency are preferably separated to such an extent that they present non-overlapping frequency bands. The specific choices of values of the inductance of the inductor 11 and the capacitances of the capacitor 12 and the moisture sensitive part 13 are preferably chosen such that the second resonance frequency falls within an appropriate license free frequency band and the first resonance frequency falls outside of this frequency band.

Returning to FIG. 1, the antenna 7 is provided at the bed 2. The antenna 7 may be provided under a mattress of the bed 2. Alternatively, the antenna 7 may be provided under an overlay mattress of the bed 2.

Alternatively, the antenna 7 may be provided under a sheet or a bed linen of the bed 2. For all these alternatives, the antenna 7 is preferably provided at a central location of the bed 2. The antenna 7 is thereby provided in a well-defined relation to the patient 3, and relatively proximate to the diaper 4 and the device 5. Preferably, the antenna 7 is provided in a plastic pocket. This provides protection against moisture and enables convenient and hygienic handling of the antenna 7 during making of the bed 2. Preferably, the antenna 7 is a loop antenna. However, other types of antennas may also be used depending on the choice of frequency band and environment etc. A loop antenna may be conveniently provided in the bed 2 with minimum discomfort for the patient 3.

The monitoring unit 6 is provided at the bed 2. The monitoring unit 6 may be mounted at the bed 2. By providing the monitoring unit 6 at the bed, the patient 3 may be monitored automatically and effortlessly with a minimum work load for the nursing staff. Alternatively, the monitoring unit 6 may be a portable, handheld unit which may be provided at the bed 2 by the nursing staff at regular intervals. The monitoring unit 6 includes transmitter circuitry and is arranged to transmit test signals to the device 5 at regular intervals (e.g. once every minute or every two minutes) and receive response signals from the device 5. The device 5 thus acts as a transponder. The monitoring unit 6 may comprise a waveform generator for generating signals to be transmitted, a signal analysis part for analyzing received signals, a DAC or other suitable circuit element for converting digital signals from the waveform generator to analog signals, and an ADC or other suitable circuit element for converting received analog signals to digital counterparts.

The monitoring unit 6 is arranged to generate and transmit a test signal. The test signal comprises a train of pulses. Preferably, the center frequency and the pulse length of the test signal is chosen such that the second resonance frequency is included in the bandwidth of the test signal. I.e. the test signal comprises a frequency component at the second resonance frequency.

According to the first example, the pulse length of the test signal is such that the first resonance frequency of the resonance circuit 10 lies well outside the frequency band of the test signal. The resonance circuit 10 may hence resonate strongly in response to the test signal when the moisture detector means 3 is sufficiently moist.

The monitoring unit 6 is further arranged to listen for and detect incoming signals from the device 5. The monitoring unit 6 is further arranged to generate an indication if a signal indicating moisture is received from the device 5. The indication may comprise activating a visual or audible indicator of the monitoring unit 6. Optionally, the monitoring unit 6 may comprise a wire based or wireless communication device (e.g. Bluetooth, ZigBee, WLAN, GPRS, 3G, optical device, sound based device etc.) for transmitting a message to a networked computer or other remote data collecting device for further analysis and storage.

Additionally, the monitoring unit 6 may be configured for determining if the patient is present within a detection range of the monitoring unit 6 and the device 5 based a detected response signal from the device. In one example, a lack of response signals detected by the monitoring unit 6 from the device 5 may be an indication that the patient is not present within the detection range. Alternatively, that the detected signal is not one of the expected response signals from the device 5.

If the monitoring unit 6 detects that the patient is not present, the monitoring unit 6 may be configured to generate and transmitting an indication, such as an alarm signal. The indication may comprise activating a visual or audible indicator of the monitoring unit 6 or at a receiving unit at a different location. The receiving unit may be a computer, a mobile unit, a mobile phone, etc. in the presents of a staff member, such as a care taker. In some alternative and/or additional examples, the monitoring unit may transmit a signal to a receiving unit to indicate that the patient is present.

Additionally, in some examples, the alarm signal may start a surveillance system which may be configured for sending a verifying signal, such as an image signal and/or a sound signal, to the receiving unit. The surveillance system may be a camera and/or a microphone, and/or other sensors arranged next to the paten. The surveillance system may be used to further verify that the patient is not present, for example before staff is sent to the location of the patient.

Alternatively, in some examples, the staff member receiving the alarm signal may choose to switch on the surveillance system instead of the surveillance system being automatically switched on by the alarm signal.

The monitoring unit 6 is further arranged to remove or at least decrease residual oscillations in the antenna 7. In more detail, the monitoring unit 6 is arranged to transmit a calibration signal prior to transmitting the test signal. After having transmitted the calibration signal, the monitoring unit 6 measures any residual signal oscillations present in the antenna 7 and based on this information configures its transmitter circuitry to adapt the test signal to obtain an effective attenuation of residual oscillations during the measurement phase. This increases the sensitivity and reliability of the detection.

The monitoring unit 6 is arranged to listen for a response to the test signal. The monitoring unit 6 is arranged to receive and sample incoming signals. As have been previously described, the amplitude of a response signal generated by the device 5 decays exponentially. The monitoring unit 6 is arranged to determine the amplitude envelope of a received signal and compare it to a predetermined value or envelope (e.g. an expected envelope based on a theoretical model or previous measurements). Thereby it may be determined whether the received signal originates from the device 5 or from some other source. For example, the monitoring unit 6 may be arranged to determine the exponent of the envelope and determine whether the exponent of the received signal is within a predetermined or expected range.

According to the first example, the monitoring unit 6 is further arranged to, in response to a positive envelope determination, determine a frequency of the response signal. If the frequency of the response signal corresponds to or matches the detection frequency (which may be the second resonance frequency), the detection signal is generated.

The frequency may e.g. be determined by performing a Fourier analysis on the received signal, by passing the signal through a band-pass filter centered at the detection frequency or by counting the number of peak values during a time interval, or similar.

By both determining the envelope and the frequency, false detection due to noise at the detection frequency may be effectively avoided.

According to an alternative example, the envelope determination may be carried out after the frequency determination.

If only a small amount of moisture is released into the diaper 4, the amount of charge carriers present at the two sets of conductors 14 and 14′ may not be sufficient for shifting the resonance frequency to the detection frequency. In that case no detection signal will be generated.

Optionally, the monitoring unit 6 may be arranged to compare the signal level of received signals with a threshold to suppress noise signals and possible weak response signals from the device 5. This reduces the risk of false detections.

According to a further option the monitoring unit 6 may be arranged to generate a detection signal if the difference between the determined frequency of the response signal and the detection frequency is smaller than a frequency threshold value. A moist condition may thus be detected if a frequency of a response signal is sufficiently close to the detection frequency.

In the above reference has been made to “a dry condition” and “a moist condition”. In general, there will always be some moist present in the air and consequently also in the diaper 4 and at the device 5. This is especially true in a diaper worn by a patient since there will also be some moisture released through perspiration. Thus, “a dry condition” is to be interpreted as corresponding to a level of moisture in the diaper 4 or at the device 5 when soiling (e.g. urination) has not occurred. Moreover, “a moist condition” is to be interpreted as corresponding to a level of moisture in the diaper 4 or at the device 5 wherein soiling (e.g. urination) has occurred.

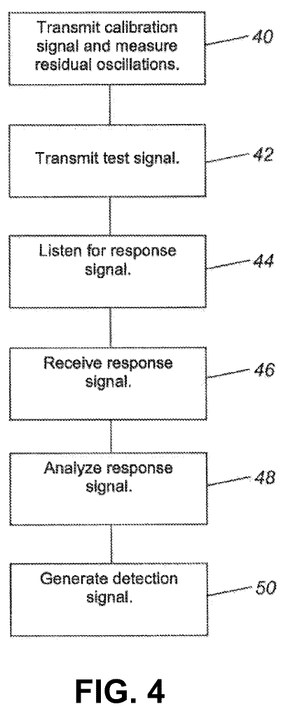

With reference to FIG. 4, a method for detecting moisture in a diaper 4 in accordance with the first example will now be described. The monitoring unit 6 transmits a calibration signal, measures residual oscillations in the antenna 7 generated by the calibration signal, and then configures its transmitter circuitry accordingly, as have been previously described. Then the monitoring unit 6 transmits the test signal (box 42).

The monitoring unit 6 then listens for a response from the device 5 (box 44). If the moisture sensitive part 13 is in a moist condition the resonance circuit 10 will be excited by the test signal and start to oscillate at its resonance frequency. This oscillation generates a response signal which is received at the monitoring unit 6 (box 46).

The monitoring unit 6 analyses the response signal (box 48). An envelope of the received signal is determined and compared to a reference envelope. Furthermore, a frequency of the response signal is determined. If the response signal presents an expected envelope and the determined frequency matches the detection frequency, the monitoring unit 6 generates a detection signal indicating that moisture has been detected (box 50). If either the envelope determination or the frequency determination is negative, no indication is generated.

Preferably the method is repeated at a suitable periodicity, e.g. every 2 minutes or every 5 minutes.

In some examples of the method, the monitoring unit 6 may be configured for determining if a patient is present based on the response signal. For example a lack of detected signal or a none expected response signal may indicate that the patient is outside a range of the monitoring unit 5 and the device 5. Hence may not be present at the location.

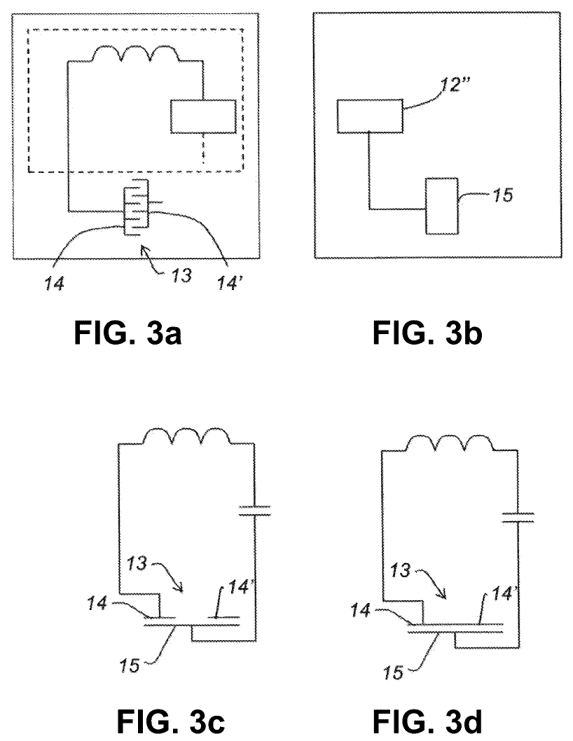

FIGS. 3a-d illustrate an alternative design of the device 5 and the resonance circuit 10. According to this alternative design, the first set of conductors 14 is connected to the inductor and the conducting layer 15 is connected to the second capacitor plate 12′. The second set of conductors 14′ is not galvanically connected to the resonance circuit 10. Hence, in a dry condition the capacitance of the moisture sensitive part 13 will mainly be determined by the capacitive coupling between the conducting layer 15 and the first set of conductors 14 as illustrated in FIG. 3c.

However, in a moist condition any charge carriers present in the moisture (and/or provided at the two sets of conductors 14 and 14′ and dissolved by the moisture) will give rise to alternating current conducting paths between the two sets of conductors 14 and 14′. The two sets of conductors 14 and 14′ may thus form an enlarged joint conductor as illustrated in FIG. 3d. The capacitance of the moisture sensitive part 13 will hence be determined by the capacitive coupling between the conducting layer 15 and the enlarged joint conductor. Since the area of the enlarged joint conductor is larger than the area of the first set of conductors 14, the capacitance of the moisture sensitive part 13 will increase. Since the moisture sensitive part 13 is connected in series with the inductor 11 and the capacitor 12 this area increase will decrease the resonance frequency of the resonance circuit 10.

According to yet another alternative design, the first set of conductors 14 may be connected to the capacitor 12 and the conducting layer 15 may be connected to the inductor 11.

A device 5 comprising a resonance circuit 10 of any of these alternative designs may hence be used in the moisture detection method of the first example.

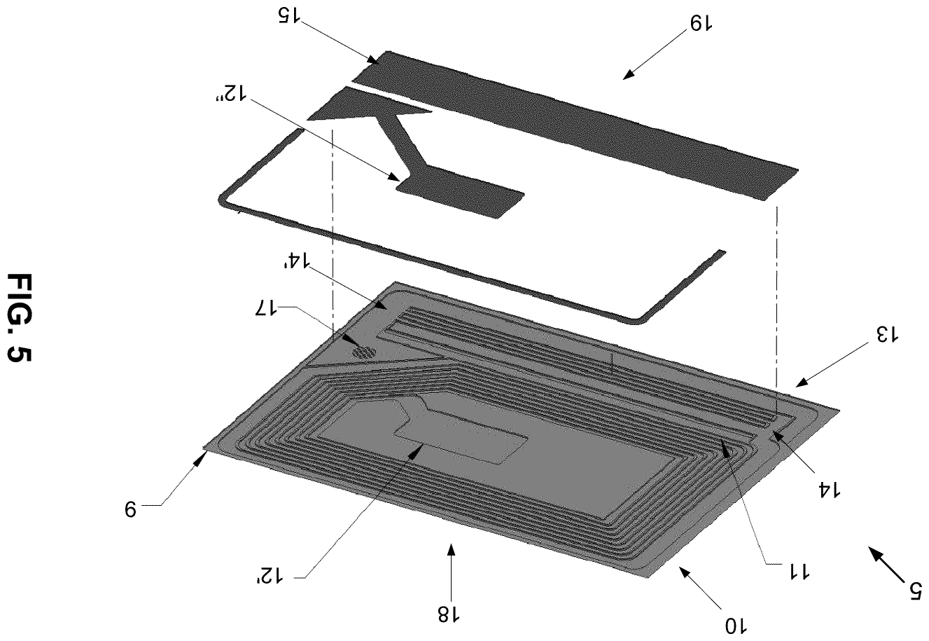

FIG. 5 is illustrating an example of a device 5, such as a RFID tag, for example a passive RFID tag. The illustrated device may be made from two etched layers, but other examples such as printed 1 layers may be possible, a first layer 18, such as a top layer that may facing the patient, and a second layer 19, such as a bottom layer that may facing the absorbent article. The two layers 18, 19 are arranged on opposite sides of an insulation layer 9. The resonance circuit 10 includes an inductor 11 and a capacitor which may be formed by two conducting surfaces, a first conducting surface 12′ of the first layer 18 and a second conducting surface 12′' of the second layer 19. The inductor is made as a loop around the resonance circuit 10. The inductor 11 may have a rectangular shape, but other shapes, such as a square, circle, elliptic, triangle etc may be possible. The capacitor formed by the two conducting surfaces 12′, 12″ may be located inside the inductor 11.

The moisture sensitive part 13 includes two sets of conductors 14 and 14′ provided as part of the first layer 18 on the insulating layer 9. The two sets of conductors 14, 14′ may be form as an interdigital finger structure. The conducting fingers of the two sets of conductors 14, 14′ are provided in parallel to each other, and in some examples parallel to a longer side of the rectangularly shaped resonance circuit 10. The two sets of conductors 14 and 14′ are separated by an insulator, such as air or a dielectric. The number of fingers, the length of the fingers and the separation of the fingers may be varied depending on e.g. the specific application, the sensitivity of the device 5 or the size of the device 5 etc.

The moisture sensitive part 13 further comprises a conducting layer 15 provided as part of the second layer 19 on the opposite side of the insulating layer 9. The conducting layer 15 is provided opposite to the two sets of conductors 14, 14′. The conducting layer 15 thus directly faces at least a part of the two sets of conductors 14, 14′, and together with the two sets of conductors 14, 14′ forms two capacitors. The two sets of conductors 14 and 14′ are connected to the resonance circuit 10. More specifically, the first set of conductors 14 is connected to the inductor 11. The second set of conductors 14′ is connected to the capacitor plate 12″. The conducting layer 15 is not galvanically connected to the resonance circuit 10. By providing the conducting layer 15 opposite to the two sets of conductors 14 and 14′, the conducting layer 15 may be capacity-coupled to each of the sets of conductors 14 and 14′.

The two layers 18, 19 are connected by some means 17, for example crimping.

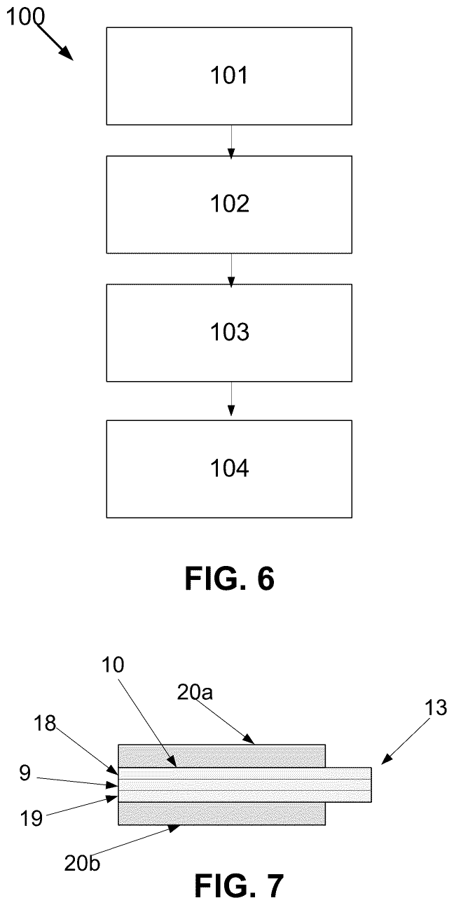

FIG. 6 is illustrating a flow chart 1 over a method 100 of manufacturing a device for detecting moisture at an absorbent article arrangeable to be attached on the absorbent article. The method provides a moisture barrier to protect the resonance circuit while leaving the moisture sensitive part exposed. The method 100 comprising the step of, providing 101 a plurality of circuits arranged side by side to form a string or strip of circuits. The plurality of circuits may be provided on a roll. Each circuit of the plurality of circuits are a herein described device 5, and includes a resonance circuit and a moisture sensitive part formed by two conducting layers arranged on opposite sides of an insulating layer. The method 100 further includes providing 102 at least one strip of a moisture barrier material. The moisture barrier may be provided on a roll. Attaching 103 the moisture barrier material to both sides of the plurality of circuits, so that the resonance circuit is covered by the moisture barrier material but not the moisture sensitive part of each circuits, and forming 104 single devices by separating the covered circuits.

In some examples may the method include folding a single strip of moisture barrier material over an edge of the plurality of circuits. Hence a single strip of moisture barrier material may cover both sides of the circuits.

In some examples may the method include providing two strips of moisture barrier material, and attaching one of the two strips to one side of the plurality of circuits and the other strip to an opposite side of the plurality of circuits. Hence the resonance circuit may be covered by the moisture barrier material but not the moisture sensitive part.

In some examples may the at least one strip of moisture barrier material be attached to the plurality of circuits using glue or an adhesive material. The adhesive material may in some examples be a double-sided bonding tape provided between the plurality of circuits and the at least one strip of moisture barrier material. In some other examples is the at least one strip of moisture barrier material attached by heating the moisture barrier material so that it's at least partially melt.

In some examples may the adhesive material be provided as a separate strip or tape of material applied between the plurality of circuits and the at least one strip of a moisture barrier material simultaneously, or sequentially. For example, may the adhesive material first be attached to either the plurality of circuits, or to the moisture barrier material, before the moisture barrier material is arranged on the resonance circuit. In other examples are they attached simultaneously by arranging the moisture barrier material on the plurality of circuits while the adhesive material is positioned in between the plurality of circuits and the moisture barrier material. In some examples may the moisture barrier material be a plastic film, such as white cell foam or sealed air plastic film.

In some examples may a further layer of fabric be attached on top of the moisture barrier material for covering, at least the side of plurality of devices facing the patient to minimize skin irritation, before the devices are separated. In some other examples is the further layer of fabric attached to the devices after they have been separated into single devices.

In some examples may a further layer of adhesive material, such as double-sided bonding tape, be applied to a side of the plurality of devices facing the absorbent article, before the devices are separated.

FIG. 7 is illustrating a cross-section of a device manufactured according to the method described herein. The resonance circuit 10 will be sandwiched between two layers of moisture barrier 20a, 20b which will protect the resonance circuit from moist and leaving the moisture sensitive part 13 exposed.

Further, some parts of the disclosure relates to detection of the presence of a patient. In particular detection of the presence of a patient in a bed using moisture detection. More specifically, the present concept may relate to detection of the presence of a patient in a bed using a system and a method for detecting moisture at an absorbent article.

The current solutions are often separate solutions that are expensive and may be considered intrusive for patient. Thus, there is a need in the prior art for a convenient, reliable and economical solution for detecting the presence of a patient. The above method for method of manufacturing may be used for manufacturing the detection system used for detection of the presence of a patient.

The detection system for detecting a presence of a patient may include a device for detecting moisture at an absorbent article arranged to be attached on the absorbent article and may include a resonance circuit including a moisture sensitive part. The resonance circuit may have at least a first resonance frequency when the moisture sensitive part is in a dry condition and at least a second resonance frequency when the moisture sensitive part is in a moist condition. The system may further include a monitoring unit arranged to transmit a test signal to the device, and determine if the patient is present based a response signal from the device.

In some examples, a lack of response signals detected by the monitoring unit may indicate that the patient is not present.

In some examples, the monitoring unit may be configured for transmitting an alarm signal to a receiving unit based on a determination that the patient is not present.

In some examples, the alarm signal may start a surveillance system configured for sending a verifying signal, such as an image signal and/or a sound signal, to the receiving unit.

In some examples, the moisture sensitive part may include a first capacitance in the dry condition and a second capacitance in the moist condition.

In some examples, the moisture sensitive part may include two conductors separated by an insulator.

In some examples, the resonance circuit may include an inductor and a capacitor.

In some examples, the test signal may include at least one pulse.

In some examples, the monitoring unit may be arranged to determine an envelope of the response signal, and to generate the detection signal if the envelope matches a reference envelope and if the determined frequency corresponds to the second resonance frequency.

The disclosure also relates to a method of detecting a presence of a patient. The method may include arranging a moisture detecting device on an absorbent article. The device may include a resonance circuit including a moisture sensitive part. The resonance circuit may have a first resonance frequency when the moisture sensitive part is in a dry condition and a second resonance frequency when the moisture sensitive part is in a moist condition. The method may further include transmitting a test signal to the device and receiving a response signal from the device. The present of the patient may be determined based on the response signal.

A part of the disclosure may relate to a mattress cover which may include an antenna for detection of a resonance frequency from a device. In particular the mattress cover may be used in relation to moisture detection. More specifically, the present concept relates to a system and a method which includes the mattress cover wherein the system and method may be for detecting moisture at an absorbent article. The mattress cover and the related system may be described as a method of manufacturing the mattress cover and system, where the described arrangement of the different features in the mattress cover and the system may be seen as method steps.

A mattress cover for detecting a resonance frequency from a device is described. In particular a device for detecting moisture at an absorbent article arranged to be attached on said absorbent article. The mattress cover may include at least two layers of fabrics, an antenna arranged between the two layers of fabrics, and an interface to connect the antenna to a monitoring unit for transmitting a test signal from the antenna to the device and for receiving a response signal from the device detected by the antenna. The antenna may be arranged in such way that it may reduce the effect of interference of magnetic fields between different parts of the antenna.

In some examples, the layers may be arranged to provide channels for the antenna to be arranged in, such as the channels are provided by welding or gluing the layers. In some examples, the antenna may include at least two parallelogram loops made from conductors. The conductors may be, wires, leads, cables etc.

In some examples, the parallelograms may be arranged diagonally to a length and/or a width direction of the mattress cover.

In some examples, at least a part of the two parallelogram loops may be overlapping, such as a third, smaller parallelogram is provided.

In some examples, the parallelogram loops may be rectangles.

In some examples, the mattress cover may include a sensor for detecting the weight of a user, such as a strain gauge arranged between the two layers of fabric. The strain gauge may be connected to the same interface to be connected to the monitoring unit.

In some examples, the mattress cover may include a sensor for detecting the temperature of a user. The temperature sensor may be connected to the same interface to be connected to the monitoring unit.

A system for detecting moisture at an absorbent article is described. The system may comprise a device arranged to be attached on the absorbent article and may include a resonance circuit including a moisture sensitive part. The resonance circuit may have a first resonance frequency when the moisture sensitive part is in a dry condition and a second resonance frequency when the moisture sensitive part is in a moist condition. The system may further include a monitoring unit including a mattress cover as described herein arranged to transmit a test signal to the device, and to receive a response signal from the device. The monitoring unit may be configured to determine a frequency of the response signal, and generate a detection signal if the determined frequency corresponds to the second resonance frequency.

A method of providing a mattress with an antenna for detecting a resonance frequency from a device is described. The device may be a device for detecting moisture at an absorbent article arranged to be attached on the absorbent article. The method may include arranging the antenna between at least two layers of fabrics. The antenna may be arranged to reduce the effect of interference of magnetic fields between different parts of the antenna.

The advantages of the described mattress cover is that it's prevents dead zones or reduces the effect of such dead zones. A dead zone is where the antenna would be less effective in sending and receiving signals to and from the device of detecting moist in an absorbent article due to, for example, no coverage between loop antennas, which may be due to interference of the magnetic fields.

The disclosed mattress over provides a cheap and effective solution.

The example herein is for a mattress cover but could equally be applicable for other bed type linings, such as at bed sheets, duvets. It could also be arranged in blankets or in a thin mattress.

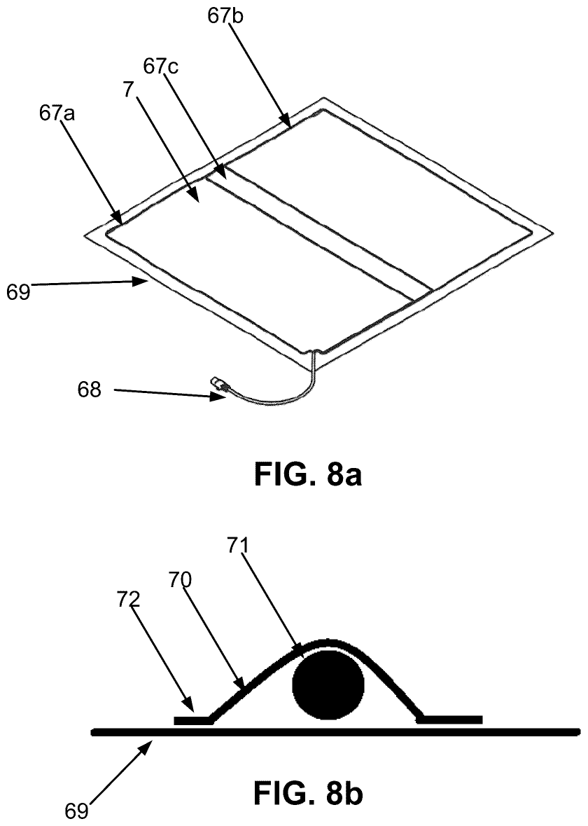

FIG. 8a is illustrating an example of an antenna 7 as part of a mattress cover 69. FIG. 8a is illustrating the bottom side of the mattress cover 69. The mattress cover can be either the size of the bed or a smaller sheet to be arranged in the area of a bed where the pelvis rest and where an absorbent article most likely will be positioned.

The mattress cover 69 may have members, such as strips, ribbons, Velcro, etc. for assisting in securing the mattress cover 69 at its position.

The mattress cover 69, is preferably made from a slip protection material, such as PU laminated textile.

The antenna 7 in the illustrated example is made from two loop antennas 67a, 67b. In other examples of the antenna 7, further loops antennas may be used, such as 3, 4, 5, 6, 7 etc. In the illustration, the loop antennas 67a, 67b are parallelograms, such as rectangles, but other shapes such as circular, elliptical or shaped as an oblong. The parallelogram, such as a rectangle, covers a large area with little discomfort. However, other types of antennas may also be used depending on the choice of frequency band and environment etc. The antennas 7 may be made from conductors, for example, wires, leads, cables etc. To connect the antenna to the monitoring unit, the antenna is equipped with a connecting interface 68.

The antenna 7 has to be arranged to provide good coverage and to avoid dead zones where the device for detecting moist is most likely located when a patient uses the bed. Dead zones mean that the antenna 7 would not be able to send a signal to, or receive a signal from the device for detecting moist. In the illustrated example, the at least a part of the two antenna loops 67a, 67b are overlapping, thereby creating an third area 67c, which in this example has the shape of a smaller parallelogram, such as a rectangle. By overlapping the two loop antennas 67a, 67b a space between the two loop antennas 67a, 67b are avoided. A space between the two loop antennas 67a, 67b may prevent a signal to be sent to, or receive from the device for detecting moist, should the device be located in a position within this dead zone.

When having loop antennas arranged close or overlapping, as illustrated in FIG. 8a there may be interference in the magnetic field that is destructive and may reduce or even cancel the signal, i.e. a dead zone. The distance between adjacent loop antennas, and the direction of the magnetic fields, therefore has to be adjusted to avoid these dead zones. In some examples, even constructive interference may be obtained.

FIG. 8b is illustrating a channel for arranging the antenna 7 in. The channel may be obtained by using a second layer 72 which is then fastened to the first layer 69, for example by being glued, welded, stitched etc. The fastening is done at two positions 72 at either side of the channel. Second layer 70 may be arranged either on top of the conductor 71 of the antenna 7, or the conductor 71 may be arranged in the channel after the second layer 70 has been fastened or attached to the first layer 69.

In some examples, not illustrated, the second layer 70 may be a single sheet of fabric. In some other examples, the second layer of fabric 70 may only have the width required to provide the channel, as illustrated in FIG. 8b. The second layer 70, may then be made from a plurality of pieces or from a single piece, such as a mask, having the shape of the antenna 7.

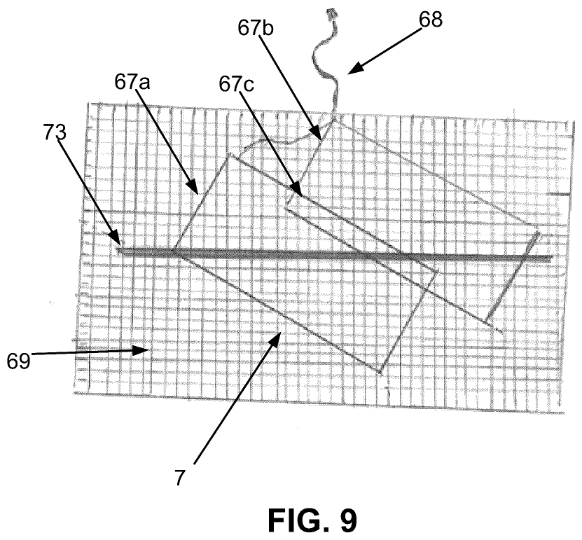

FIG. 9 is illustrating a further example of the arrangement of loop antennas 67a, 67b. In the illustrated example, loop antennas 67a, 67b are arranged diagonally to a length and/or a width direction of the mattress cover. This may improve the coverage and decrease the likelihood that there may be a dead zone due to interference of the magnetic fields in the overlapping area 67c. Even if there would be a dead zone in the overlapping area 67c, the effect of such interference may be reduced due to the diagonally arrangement. The diagonally arrangement improves the likelihood that even if there is a dead zone in the overlapping area 67c, the device for detecting moist would most likely be located in relation to the antenna 7 in a way that a signal may still be transmitted to the device from the antenna 7, or from the device to the antenna 7.

In some examples, the mattress cover may include a sensor. The sensor may for example be used for detecting the weight and/or weight changes of the patient. An example of a sensor for detecting the weight and/or a weight change of a patient is a strain gauge 73. Alternatively, to using a separate strain gauge, the antenna may be used as a strain gauge. The strain gauge 73 may be connected to the interface 68 to be connected to the monitoring unit. The weight or weight changes may give important information about a patient to a care taker, such as a nurse.

In one further examples, the mattress cover may include a sensor for detecting the temperature of a user, such as a patient, not illustrated. The temperature sensor is connected to the interface 68 to be connected to the monitoring unit. The temperature may provide important health information about a patient to a care taker, such as a nurse. If the temperature goes down it may indicate that the patient is dead. An alarm signal may then be transmitted from the monitoring unit to a receiving unit in a similar way as described herein in relation to method of detecting the presence of a patient.

In the above, the invention has mainly been described with reference to a few examples. However, as is readily appreciated by a person skilled in the art, other examples than the ones disclosed above are equally possible within the scope of the inventive concept, as defined by the appended claims.

1. A mattress cover for detecting a resonance frequency from a device for detecting moisture at an absorbent article, the device is configured to be attached on the absorbent article, the mattress cover comprises at least two layers of fabrics, an antenna arranged between the two layers of fabrics, and an interface to connect the antenna to a monitoring unit for transmitting a test signal from the antenna to the device for detecting moisture and for receiving a response signal from the device for detecting moisture detected by the antenna; and
wherein the antenna comprises at least two parallelogram loops made from conductors arranged to reduce the effect of interference of magnetic fields between different parts of the antenna.

2. The mattress cover of claim 1, wherein the layers are arranged to provide channels for the antenna to be arranged in.

3. The mattress cover of claim 2, wherein the channels are provided by welding or gluing the layers.

4. The mattress cover of claim 1, wherein said conductors are wires.

5. The mattress cover of claim 1, wherein the parallelograms are arranged diagonally to a length and/or a width direction of the mattress cover.

6. The mattress cover of claim 5, wherein at least a part of the two parallelogram loops are overlapping.

7. The mattress cover of claim 6, wherein the overlapping provides as a third, smaller parallelogram.

8. The mattress cover of claim 6, wherein the parallelogram loops are rectangles.

9. The mattress cover of claim 1, wherein the mattress cover includes a sensor for detecting the weight of a user, such as a strain gauge arranged between the two layers of fabric.

10. The mattress cover of claim 1, wherein the mattress cover includes a sensor for detecting the temperature of a user.

11. The mattress cover of claim 1, wherein one of the at least two layers is a top layer comprising a breathable textile material, and one of the at least two layers is a bottom layer comprising a waterproof material.

12. A system for detecting moisture at an absorbent article, the system comprising:
a device for detecting moisture arranged to be attached on the absorbent article and comprising a resonance circuit having a first resonance frequency when the absorbent article is in a dry condition;
a monitoring unit comprising a mattress cover configured to:
transmit a test signal to the device for detecting moisture;
receive a response signal from the device detecting moisture;
determine a frequency of the response signal; and
generate a detection signal if the determined frequency corresponds to the first resonance frequency.

13. The system of claim 12, wherein the resonance circuit and a moisture sensitive part are formed by two conducting layers arranged on opposite sides of an insulating layer.

14. The system of claim 13, wherein the resonance circuit assumes the first resonance frequency when the moisture sensitive part is in a dry condition and a second resonance frequency, which is different from the first resonance frequency, when the moisture sensitive part in a moist condition.

15. The system of claim 14, wherein the monitoring unit generates the detection signal if the determined frequency corresponds to the moist condition.

16. A system for detecting moisture at an absorbent article, the system comprising:
a device for detecting moisture arranged to be attached on the absorbent article and comprising a resonance circuit having a first resonance frequency when the absorbent article is in a moist condition;
a monitoring unit comprising a mattress cover configured to:
transmit a test signal to the device for detecting moisture;
receive a response signal from the device detecting moisture;
determine a frequency of the response signal; and
generate a detection signal if the determined frequency corresponds to the first resonance frequency.

17. The system of claim 16, wherein the resonance circuit and a moisture sensitive part are formed by two conducting layers arranged on opposite sides of an insulating layer.

18. The system of claim 17, wherein the moisture sensitive part provides at least a second resonance frequency when the moisture sensitive part is in a dry condition.

19. The system of claim 17, wherein the monitoring unit generates the detection signal if the determined frequency corresponds to the dry condition.
